SEQUENCE LISTING (59 sequences)

SEQ ID NO: 1 - DNA, 35 bp, Artificial Sequence
  Primer
atttttgtgaaaagttttgtcaatgtagttcagag

SEQ ID NO: 2 - DNA, 20 bp, Artificial Sequence
  Primer
tcacactctcggacagccag

SEQ ID NO: 3 - DNA, 23 bp, Artificial Sequence
  Primer
caacaccaagcagtgcccgtgct

SEQ ID NO: 4 - DNA, 24 bp, Artificial Sequence
  Primer
tgcagttgcttcagtacccataat

SEQ ID NO: 5 - DNA, 26 bp, Artificial Sequence
  Primer
atccccgtgtactttcccatcataat

SEQ ID NO: 6 - DNA, 21 bp, Artificial Sequence
  Primer
ttcctctttgcatggaatttg

SEQ ID NO: 7 - DNA, 20 bp, Artificial Sequence
  Primer
agaggagtgggggaagagtc

SEQ ID NO: 8 - DNA, 20 bp, Artificial Sequence
  Primer
tggacgatgtgctctatgcc

SEQ ID NO: 9 - DNA, 20 bp, Artificial Sequence
  Primer
ggatggtgatggtttggtag

SEQ ID NO: 10 - DNA, 22 bp, Artificial Sequence
  Primer
tctgtaaggcccaaagtgggta

SEQ ID NO: 11 - DNA, 25 bp, Artificial Sequence
  Primer
acctcagcatgacaatatgtcacaa

SEQ ID NO: 12 - DNA, 20 bp, Artificial Sequence
  Primer
ccgacacagacaccatcaac

SEQ ID NO: 13 - DNA, 20 bp, Artificial Sequence
  Primer
cttttctgcccaacttctgc

SEQ ID NO: 14 - DNA, 20 bp, Artificial Sequence
  Primer
ggaggagagcggtggtcaaa

SEQ ID NO: 15 - DNA, 21 bp, Artificial Sequence
  Primer
tgtgcagctggctcaagtcaa

SEQ ID NO: 16 - DNA, 20 bp, Artificial Sequence
  Primer
tgcaccaccaactgcttagc

SEQ ID NO: 17 - DNA, 21 bp, Artificial Sequence
  Primer
ggcatggactgtggtcatgag

SEQ ID NO: 18 - DNA, 22 bp, Artificial Sequence
  Primer
gtactgtgataactgaaggatg

SEQ ID NO: 19 - DNA, 22 bp, Artificial Sequence
  Primer
attctgcttctctttgccttgt

SEQ ID NO: 20 - DNA, 21 bp, Artificial Sequence
  Primer
gactgaactgtcctttttcgg

SEQ ID NO: 21 - DNA, 20 bp, Artificial Sequence
  Primer
cctttctcaatgtgatggca

SEQ ID NO: 22 - DNA, 20 bp, Artificial Sequence
  Primer
ctaatgcattgccttcagca

SEQ ID NO: 23 - DNA, 20 bp, Artificial Sequence
  Primer
ttagcatcttgggctcacct

SEQ ID NO: 24 - DNA, 18 bp, Artificial Sequence
  Primer
acctggtggcaggaacac

SEQ ID NO: 25 - DNA, 18 bp, Artificial Sequence
  Primer
tgaggcgctgctataccc

SEQ ID NO: 26 - DNA, 19 bp, Artificial Sequence
  Primer
caggcgatggcctaagact

SEQ ID NO: 27 - DNA, 20 bp, Artificial Sequence
  Primer
cagggtaagtctgggactgc

SEQ ID NO: 28 - DNA, 21 bp, Artificial Sequence
  Primer
tgaagactctggggacttttg

SEQ ID NO: 29 - DNA, 25 bp, Artificial Sequence
  Primer
aatatacacagaggattgcttagcc

SEQ ID NO: 30 - DNA, 20 bp, Artificial Sequence
  Primer
tggctggttgcatatgtagg

SEQ ID NO: 31 - DNA, 21 bp, Artificial Sequence
  Primer
tggtgtttctggtgtctgtca

SEQ ID NO: 32 - DNA, 22 bp, Artificial Sequence
  Primer
aaaacaaatgttgcaatccaaa

SEQ ID NO: 33 - DNA, 21 bp, Artificial Sequence
  Primer
tgcaaataacaagatgcctga

SEQ ID NO: 34 - DNA, 22 bp, Artificial Sequence
  Primer
actgagccattgagggtaccta

SEQ ID NO: 35 - DNA, 18 bp, Artificial Sequence
  Primer
ccccagaaggcaaaggat

SEQ ID NO: 36 - DNA, 21 bp, Artificial Sequence
  Primer
gtgagacagccaatggagaac

SEQ ID NO: 37 - DNA, 22 bp, Artificial Sequence
  Primer
tgttcgttaggcaacagctaca

SEQ ID NO: 38 - DNA, 20 bp, Artificial Sequence
  Primer
cagcctttgatggtgtgtgt

SEQ ID NO: 39 - DNA, 21 bp, Artificial Sequence
  Primer
tccattactggagactctggg

SEQ ID NO: 40 - DNA, 25 bp, Artificial Sequence
  Primer
ttagctggctctgagtatgaataac

SEQ ID NO: 41 - DNA, 20 bp, Artificial Sequence
  Primer
gctggaaaacccaacttctg

SEQ ID NO: 42 - DNA, 26 bp, Artificial Sequence
  Primer
gtgactttatgcccctttagagacaa

SEQ ID NO: 43 - DNA, 26 bp, Artificial Sequence
  Primer
gaagggctacaactggaactggaata

SEQ ID NO: 44 - DNA, 20 bp, Artificial Sequence
  Primer
attcctccaggtgggtgtgg

SEQ ID NO: 45 - DNA, 21 bp, Artificial Sequence
  Primer
ctaagccgctgttgccttggc

SEQ ID NO: 46 - DNA, 18 bp, Artificial Sequence
  Primer
tcctggcccggggcctgg

SEQ ID NO: 47 - DNA, 19 bp, Artificial Sequence
  Primer
ctgttccccgggctcccgc

SEQ ID NO: 48 - DNA, 19 bp, Artificial Sequence
  Primer
tgtcccgggatcctcttct

SEQ ID NO: 49 - DNA, 21 bp, Artificial Sequence
  Primer
tagagacgttggtgcggaaat

SEQ ID NO: 50 - DNA, 20 bp, Artificial Sequence
  Primer
tagagggtcaccgcgtctat

SEQ ID NO: 51 - DNA, 20 bp, Artificial Sequence
  Primer
tcacaggtgctttgcagttc

SEQ ID NO: 52 - DNA, 20 bp, Artificial Sequence
  Primer
gtttctgctacgctgctgct

SEQ ID NO: 53 - DNA, 20 bp, Artificial Sequence
  Primer
caccagctcacttaccacca

SEQ ID NO: 54 - DNA, 20 bp, Artificial Sequence
  Primer
tactagcggttttacgggcg

SEQ ID NO: 55 - DNA, 24 bp, Artificial Sequence
  Primer
tcgaacaggaggagcagagagcga

SEQ ID NO: 56 - DNA, 20 bp, Artificial Sequence
  Primer
ctaggagggtggaggtaggg

SEQ ID NO: 57 - DNA, 20 bp, Artificial Sequence
  Primer
gccccaaacaggagtaatga

SEQ ID NO: 58 - DNA, 22 bp, Artificial Sequence
  Primer
cagtgaggtctcagcatcagca

SEQ ID NO: 59 - DNA, 22 bp, Artificial Sequence
  Primer
ctggaggctatgggggtacttg

(19) United States
(12) Patent Application Publication
(10) Pub. No.: US 2011/0224284 A1
(43) Pub. Date: Sep. 15, 2011

(54) PUTATIVE TUMOR SUPPRESSOR MICRORNA-101 MODULATES THE CANCER EPIGENOME BY REPRESSING THE POLYCOMB GROUP PROTEIN EZH2

(75) Inventors: Jeffrey J. Friedman, Los Angeles, CA (US); Gangning Liang, Rowland Heights, CA (US); Peter A. Jones, La Canada, CA (US)

(73) Assignee: UNIVERSITY OF SOUTHERN CALIFORNIA, Los Angeles, CA (US)

(21) Appl. No.: 13/119,699

(22) PCT Filed: Sep. 22, 2009
(86) PCT No.: PCT/US2009/057890

§ 371 Date: May 13, 2011

Related U.S. Application Data
(60) Provisional application No. 61/099,114, filed on Sep. 22, 2008.

(51) Int. Cl. A61K 31/7088; C12N 5/02; A61P 35/00

(52) U.S. Cl. 514/44 A; 435/375

(57) ABSTRACT

The present invention relates in general to microRNA profiling in disease. More specifically, the invention provides for methods and compositions of microRNA to inhibit the growth and formation of tumors.

The present application claims the benefit of the filing date of U.S. Provisional Application No. 61/099,114 filed Sep. 22, 2008, the disclosure of which is incorporated herein by reference in its entirety.

### STATEMENT REGARDING FEDERALLY SPONSORED RESEARCH OR DEVELOPMENT

This invention was made with government support under Contract No. RO1 CA 83867 awarded by the National Institutes of Health. The government has certain rights in the invention.

### FIELD OF THE INVENTION

The present invention relates in general to microRNA. More specifically, the invention provides for microRNA that may be used in cancer therapy.

### BACKGROUND OF THE INVENTION

Polycomb group (PcG) proteins are chromatin modifying enzymes that

were discovered as homeotic regulators in Drosophila melanogaster. Subsequent work has revealed that they are important in stem cell maintenance, Xinactivation, imprinting, and development, and many PcG proteins are dysregulated in human cancer (1). The PcG protein EZH2 is the catalytic subunit of the Polycomb Repressive Complex 2 (PRC2), which includes SUZ12 (suppressor of zeste 12) and EED (embryonic ectoderm development) (2). EZH2 is a critical part of the cellular machinery involved in epigenetically regulating gene transcription (2). PRC2 represses genes by trimethylating the core histone H3 lysine 27 (H3K27me3) at and around the promoter regions of target genes (1).

EZH2 enhances neoplastic transformation (3), is overexpressed in many cancers, and is strongly associated with metastatic breast and prostate cancers (3-7). In addition, knockdown of EZH2 inhibits cancer cell proliferation (6). Recent work has shown that overexpression of EZH2 is directly responsible for the de novo suppression of multiple genes in human cancer (8, 9). However, the cause of EZH2 overexpression in cancer is not clear. Intriguingly, a significant subset of PRC2 target genes in cancer were also targets of PRC2 in embryonic stem (ES) cells (10). This illustrates a strong association between the function of PRC2 in cancer and stem cells which represent de-differentiated and proliferative cell states. Therefore, EZH2 overexpression might cause a normal cell to dedifferentiate back to a stem-cell like state by epigenetically repressing cell fate regulating genes and tumor-suppressor genes which initiates tumor development (1, 8, 11).

EZH2 was initially found to be elevated in a subset of aggressive clinically localized prostate cancers and almost all metastatic prostate cancers (6). Subsequently, EZH2 has also been found to be aberrantly overexpressed in breast cancer (3), melanoma (33), bladder cancer (34), gastric cancer (35), and other cancers (44). Thus, although EZH2 is broadly overexpressed in aggressive solid tumors and has properties of an oncogene, the genetic mechanism of EZH2 elevation in cancer is unclear.

MicroRNAs (miRNAs) are ˜22 nucleotide non-coding RNA molecules that usually function as endogenous repressors of target genes. In animals, miRNAs can bind with imperfect complementarity to the 3′ untranslated region (3′UTR) of the target mRNA via the RNA-induced silencing complex. The resulting gene repression occurs by multiple mechanisms including enhanced mRNA degradation and translational repression (12). Due to the promiscuity of miRNA binding to target mRNAs, each miRNA may control numerous genes and each mRNA may be controlled by many miRNAs (13). Developmental timing, cell death, proliferation, hematopoiesis, insulin secretion, and the immune response are just a few examples of critical biological events that depend on faithful miRNA expression (14).

An analysis of recent miRNA profiling studies in cancer revealed that miR-101 was downregulated in breast, lung, prostate, ovarian, colon and liver cancers (36), which suggests that decreased miR-101 expression may be a marker of solid tumors (37,6). Intriguingly, miR-101 can be produced from two genomic loci, miR-101-1 on chromosome 1p31 and miR-101-2 on chromosome 9p24. This complicates attempts to address the transcriptional regulation of miR-101, although loss of heterozygosity at chromosome 1p and chromosome 9p are associated with cancer. In fact, a recent report convincingly showed that genomic loss of miR-101 occurs in a significant number of prostate tumors and was associated with progression (6). Further studies will have to examine the causes of miR-101 down regulation in tumors without LOH at either miR-101 locus. However, it seems clear that the resulting up regulation of miR-101 targets, including EZH2, by decreases in miR-101 is selected for during tumorigenesis. Interestingly, miR-101 is repressed in human embryonic stem ES cells, but is upregulated during differentiation, which links the proliferative, de-differentiated states of cancer and stem cells by a common miRNA (38).

In the past few years miRNA profiling of various human cancers has revealed many miRNAs that function as tumor suppressors, such as let-7, miR-15a/16-1 and the miR-34 family, or oncogenes such as miR-155 and the miR-17-92 cluster (15, 16). In addition, miRNA profiles were better able to predict tumor type than were mRNA profiles (17). However, miRNA profiling of bladder cancer with normal and matched tumor tissues and functional studies of differentially expressed miRNAs in bladder cancer remains to be conducted. Bladder cancers in the United States are almost exclusively transitional cell carcinomas (TCC), and in 2007 TCC was the fifth most common cancer diagnosis according to the National Cancer Institute (NCI).

Therefore, the inventors sought to generate a miRNA expression profile for TCC by comparing primary TCCs to their corresponding normal urothelium. The inventors found many differentially expressed miRNAs, several of which showed putative tumor suppressor functions. The miRNA that most consistently and dramatically suppressed growth was miR-101, which the inventors confirmed can directly target EZH2 and repress H3K27me3. Furthermore, our results indicate that a significant subset of genes is regulated by both miR-101 and EZH2.

### SUMMARY OF THE INVENTION

In one embodiment, the invention relates to methods of using miRNA to inhibit cell proliferation and colony formation.

In a related embodiment, the invention relates to compositions for inhibiting cell proliferation and colony formation comprising miRNA.

In another embodiment, the invention relates to methods of using miRNA to suppress tumor growth.

In yet another embodiment, the invention relates to methods of using miRNA to inhibit tumor formation.

In accordance with another embodiment, the invention relates to methods of using miRNA to decrease EZH2 expression.

In a closely related embodiment, the invention relates to methods of increasing miRNA expression.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE FIGURES

FIG. 1. (A) Total RNA from 9 TCC samples were pooled and labeled with Cy5. Total RNA from 9 matched normal tissues were pooled and labeled with Cy3. The samples were hybridized by LC Sciences to an array with probe content from Sanger miRBase 8.0 interrogating 328 miRNAs. Plot of the Tumor signal vs. Normal signal shows all transcripts with those showing fold change>8 in black and a table shows the most differentially expressed transcripts (including miR-127) that were validated with RT-qPCR in additional patient samples. (B) miRNART-qPCR of 28 clinical TCC samples and matched normal urothelium. All reactions were done in duplicate and U6 was the internal control. The graph shows the ratio of miRNA expression of Tumor/Normal on a logarithmic scale. *indicates statistical significance, error bars are the 95% confidence interval. (C) Cell proliferation assays were conducted by transferring equal cell numbers to 10 cm dishes 48 h post-transfection with miRNA expression vectors or empty vector (e.v.) control. After 13 days under G418 selection total cells were counted and normalized to the empty vector, (D) Colony formation assays were conducted by seeding equal cell numbers 48 h post-transfection into 6-well plates. Colonies were stained and counted after 13 days under G418 selection and normalized to the empty vector control. For (C) and (D) * indicates p-value<0.02 according to Dunnet's method (except UM-UC-3 miR-145, p-value=0.044), error bars are the standard error of the mean.

RT-qPCR confirms that pcDNA3.1(+) vectors express mature miRNAs. RT-qPCR was done in duplicates on total RNA isolated 48 hours after transfection in T24 (E), UM-UC-3 (F), and TCCSUP (G) cells. The miRNA expression vectors are compared to empty vector controls and normalized to U6 levels.

FIG. 2. miR-101 is downregulated in colon and prostate tumors. RT-qPCR for miR-101 of 10 colon cancer patient sample sets and 7 prostate sets shows that miR-101 is downregulated in colon and prostate cancer. All reactions were done in duplicate, the miR-101 signal was divided by U6 and the average Normal value was normalized to 1. * p-value=0.01, ** p-value=0.046, p-values were calculated using a 1-tailed paired t-test.

FIG. 3. miR-101 directly targets EZH2. (A) The highly conserved sequence of the 3′UTR of EZH2 for human, mouse, rat, dog, and chicken are shown. The nucleotides that were mutated for the luciferase insert are marked with *. (B) Western blot analysis of TCC cell lines after transient transfection with pre-miR-101 or control precursors at a final concentration of 50 nM. These experiments used transiently transfected synthetic miRNA precursors to examine target interactions while the stably transfected pcDNA3.1(+) miRNA expression vectors examined effects on cell growth in FIG. 1. Lysates were prepared 48 h after transfection and membranes were probed with antibodies to EZH2, H3K27me3, and -actin as a loading control. Bands were quantitated by Quantity One software (Bio-Rad). (C) Luciferase assay conducted in UM-UC-3 cells 24 h after cotransfection with premiR-101, renilla luciferase vector pRL-SV40, and either the firefly luciferase reporter pGL3-control containing wild type EZH2 3′UTR insert (GUACUGU) or mutated EZH2 3′UTR insert (CUAGUCU). Relative luciferase activity was normalized to the no insert control (* p-value<0.01).

FIG. 4. pre-miR-101 transfection and siRNA to EZH2 lead to the up-regulation of overlapping genes. (A) Western blot analysis of UM-UC-3 cells transfected with siRNA to EZH2, control siRNA, pre-miR-101, or control precursors to a final concentration of 60 nM. Total protein was extracted 72 h after transfection and membranes were probed with antibodies to EZH2, H3K27me3, and -actin as a loading control. (B) Illumina Human 6 v 2 chips were used to interrogate the mRNA levels from UM-UC-3 cells 72 h after transfection with pre-miR-101, control precursors, siRNA to EZH2 and control siRNA. Based on the criteria fold change>1.5 and t-test p-value<0.05, 1,092 genes were up-regulated after premiR-101 transfection, while 105 genes were up-regulated after treatment with siRNA to EZH2. There was an overlap of 43 genes (p-value<10₋₁₁ based on hypergeometric distribution).

FIG. 5. Knockdown of EZH2 decreases cell proliferation and colony formation in TCC cell lines. (A) cell proliferation assays were conducted by transferring equal cell numbers to 10-cm dishes 48 h posttransfection with 4 different expression vectors (clones 74, 75, 76, and 77) containing distinct shRNAs to EZH2 or control shRNA vector. After 13 d under puromycin selection, total cells were counted and normalized to the empty vector. (B) colony formation assays were conducted by seeding equal cell numbers 48 h posttransfection into 10-cm dishes. Colonies were stained and counted after 13 d under puromycin selection and normalized to the control shRNA vector. *, P<0.05 according to t test; columns, mean; bars, SE. (C) photographs of representative colony formation assays.

FIG. 6. miR-101 regulates EZH2 transcript and protein expression. (A) Venn diagram displaying miRNAs computationally predicted to target EZH2 by PicTar (blue), miRanda (red), TargetScan (green), and MicroInspector (orange). (B) Schematic of two predicted miR-101-binding sites in the EZH2 3′UTR. (C) miR-101 regulates EZH2 transcript expression. Quantitative RT-PCR of EZH2 in SKBr3 cells transfected with precursor miR-101 is shown. Control miR and other precursor miRNAs (miR-26a, miR-128a, and miR-217) were also used for transfection. (D) miR-101 regulates PRC2 protein expression. miR-101 down-regulates EZH2 protein as well as PRC2 members SUZ12 and EED in SKBr3 cells. Control mills and EZH2-specific siRNA were also used for transfection. The experiment was performed three independent times and a representative result is displayed.

GAPDH, glyceraldehyde-3-phosphate dehydrogenase.

FIG. 7. The role of miR-101 in regulating cell proliferation, invasion, and tumor growth. (A) miR-101 overexpression reduces cell proliferation. A cell growth assay of SKBr3 cells treated with either precursor miR-101 or siRNA targeting EZH2 is shown. Cell growth relative to the control miRNA and control siRNA duplex was measured. Rescue experiments were performed by overexpressing EZH2 (minus its endogenous 3′UTR) in miR-101-treated cells. (B) miR-101 expression decreases cell invasion of DU145 prostate carcinoma cells. The inventors transfected cells with miR-101, EZH2-specific siRNA, control miR, and nontargeting siRNA. miR-101 was also overexpressed in those cells that overexpressed EZH2 by andenoviral infection. All cells were subjected to a matrigel invasion assay. (C) AntagomiRs to miR-101 induce the invasiveness of benign immortalized H16N2 breast epithelial cells. Representative fields of invaded and stained cells are shown in the inset. P values were calculated between control antagomiR, antagomiR 101i, and antagomiR-101ii. (D) Overexpression of miR-101 attenuates prostate tumor growth. Overexpression of miR-101 reduces DU145 tumor growth in a mouse xenograft model. Plot of mean tumor-volume trajectories over time for the mice inoculated with (red) miR-101- and (green) vector-expressing DU145 cells. Error bars represent the SE of the mean at each time point. The inset displays the decrease of EZH2 protein levels in miR-101-expressing cell lines.

FIG. 8. miR-101 regulation of the cancer epigenome through EZH2 and H3K27 trimethylation. (A) ChIP assay of the trimethyl H3K27 histone mark when miR-101 is overexpressed. Known PRC2 repression targets were examined in SKBr3 cells. ChIP was performed to test H3K27 trimethylation at the promoters of ADRB2, DAB2IP, CIITA, RUNX3, CDH1, and WNT1. GAPDH, KIAA0066, and NUP214 gene promoters served as controls. (B) Quantitative RT-PCR of EZH2 target genes was performed with SKBr3 cells transfected with miR-101. The EZH2 transcript and its known targets, including ADRB2, DAB2IP, CIITA, RUNX3, and E-cadherin (CDH1) were measured.

FIG. 9. Genomic loss of the miR-101 locus may explain overexpression of EZH2 in solid tumors. (A) miR-101 transcript levels are inversely correlated with EZH2 expression in prostate cancer progression. The inventors performed quantitative PCR for miR-101 and miR-217 by using total RNA from benign adjacent prostate, prostate cancer (PCA), and metastatic (MET) prostate cancer tissue. EZH2 expression was analyzed from the same RNA samples. (B) Genomic PCR of miR-101-1 and miR-101-2 in prostate cancer progression. Vertical axes represent log (base 2) relative quantification values; dashed lines are shown at the deletion threshold of log 2(0.7)≈−0.51. For clarity, points have been horizontally displaced within each sample class, (C) Heat-map representation of matched normal, tumor, and metastatic samples (from right to left) in which miR-101 transcript, EZH2 transcript, and both miR-101-1 and miR-101-2 relative copy number were assessed. miR-101 and EZH2 expression is represented by a color scale highlighting down-regulation (green), no alteration (black), and up-regulation (red) of transcripts. miR-101-1 and miR-101-2 relative quantitation (RQ) of copy number are represented as homozygous loss (<0.3; bright green), single-copy loss (<0.7; light green), no copy number change (≧0.7 and ≦1.3; black), single-copy gain (>1.3; light red), and double-copy gain (>1.7; bright red). (D) Evidence that the miR-101-1 locus is somatically lost in tumors samples relative to matched normal samples. Nine metastatic prostate cancers were chosen that had copy number loss in the miR-101-1 locus, and matched normal tissue was analyzed for comparison. Bar heights represent differences in log 2(RQ) values between metastatic and matched normal tissues.

### DETAILED DESCRIPTION OF THE INVENTION

In the past few years miRNA profiling of various human cancers has revealed many miRNAs that function as tumor suppressors (15, 16). In fact, it has been shown that miRNA profiles are better able to predict tumor type than mRNA profiles (17). However, miRNA profiling of bladder cancer with normal and matched tumor tissues and functional studies of differentially expressed miRNAs in bladder cancer has not been done. Therefore, the inventors have generated a miRNA expression profile for TCC by comparing primary TCCs to their corresponding normal urothelium.

As used herein the terms “cancer” and “cancerous” refer to or describe the physiological condition in mammals that is typically characterized by unregulated growth of malignant cells. Examples of cancer include but are not limited to, carcinoma, lymphoma, blastoma, sarcoma, and leukemia. More particular examples of such cancers include breast cancer, brain cancer, bladder cancer, prostate cancer, colon cancer, intestinal cancer, squamous cell cancer, lung cancer, stomach cancer, pancreatic cancer, cervical cancer, ovarian cancer, liver cancer, skin cancer, colorectal cancer, endometrial carcinoma, salivary gland carcinoma, kidney cancer, thyroid cancer, various types of head and neck cancer, and the like.

The term “overexpression,” as used herein refers to overexpression of a gene and/or its encoded protein in a cell, such as a cancer cell. A cancer cell that “overexpresses” a protein is one that has significantly higher levels of that protein compared to a noncancerous cell of the same tissue type.

The term “subject” refers to any animal, including humans, mice, rats, cats, dogs, chickens and any other animal with a highly conserved sequence of the 3′UTR of EZH2.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### Materials and Methods

### Cell Lines and Primary Tumors

T24, UM-UC-3, and TCCSUP cells were obtained from the American Type Culture Collection (ATCC) and cultured according to ATCC protocols. Patient samples were obtained though USC/Norris Tissue Procurement Core

Resource after informed consent and Institutional Review Board approval (IRB-#886005 and #926041) at the USC/Norris Comprehensive Cancer Center.
miRNA Microarray

One g of total RNA from each of 9 TCCs was pooled and the same was

done with 9 matched normal tissues. miRNA microarray analysis was done as previously described (18). Specifically, miRNA microarray analysis was carried out by LC sciences (http://www.lcsciences.com/; Houston, Tex.). Poly-A tails were added to the RNA sequences at the 30 ends using a poly(A) polymerase, and nucleotide tags were then ligated to the poly-A tails. For each dual-sample experiment, two sets of RNA sequences were added with tags of two different sequences. The tagged RNA sequences were then hybridized to the miRNA microarray chip containing 313 human miRNA probes. The probe sequences are available upon request. The labeling reaction was carried out during the second hybridization reaction using tag-specific dendrimer Cy3 and Cy5 dyes, RNAs from untreated cells and cells treated with 5-Aza-CdR and/or PBA were labeled with Cy3 and Cy5, respectively. The human miRNA chip includes seven redundancies for each miRNA. The data were corrected by subtracting the background and normalizing to the statistical median of all detectable transcripts. Background was calculated from the median of 5% to 25% of the lowest-intensity cells. The data normalization balances the intensities of Cy3- and Cy5-labeled transcripts so that differential expression ratios can be correctly calculated. All data were submitted to the ArrayExpress database, and the accession number is E-MEXP-1917,
Reverse Transcription and Taqman qPCR

miRNA Taqman assays (Applied Biosystems) were used according to the manufacturer's protocol. Specifically, reverse transcriptase reactions contained RNA samples including purified total RNA, cell lysate, or heat-treated cells, 50 nM stem-loop RT primer (P/N: 4365386 and 4365387, Applied Biosystems), 1×RT buffer (P/N: 4319981, Applied Biosystems), 0.25 mM each of dNTPs, 3.33 U/μl MultiScribe reverse transcriptase (P/N: 4319983, Applied Biosystems) and 0.25 U/μl RNase inhibitor (P/N: N8080119; Applied Biosystems). The 7.5 μl reactions were incubated in an Applied Biosystems 9700 Thermocycler in a 96- or 384-well plate for 30 min at 16° C., 30 min at 42° C., 5 min at 85° C. and then held at 4° C. All. Reverse transcriptase reactions, including no-template controls and RT minus controls, were run in duplicate.

### PCR

Real-time PCR was performed using a standard TaqMane® PCR kit protocol on an Applied Biosystems 7900HT Sequence Detection System (P/N: 4329002, Applied Biosystems). The 10 μl PCR included 0.67 μl RT product, 1× TaqMan® Universal PCR Master Mix (P/N: 4324018, Applied Biosystems), 0.2 μM TaqMan® probe, 1.5 μM forward primer and 0.7 μM reverse primer. The reactions were incubated in a 384-well plate at 95° C. for 10 min, followed by 40 cycles of 95° C. for 15 s and 60° C. for 1 min. All reactions were run in duplicate.)

### Expression Vectors

Expression vectors were made by cloning ˜200 bp surrounding the precursor miRNA into pcDNA3.1(+) (Invitrogen).

### Cell Proliferation and Colony Formation Assays

Cell proliferation assays were conducted as described previously (19). T24, UMUC3 and TCCSUP cells were seeded in 6-well dishes so that 24 hours later they were 90% confluent. They were transfected using 10 μL Lipofectamine 2000 (Invitrogen) and 4 μg plasmid according to the manufacturer's protocol.

The cell proliferation assays were conducted in triplicate as described previously (19). Each well was trypsinized and equal cell numbers were plated onto 10 cm dishes with medium containing G418 (Sigma) (T24 400 μg/mL, UMUC3 1 mg/mL, TCCSUP 1 mg/mL). Medium was changed every 3-4 days and total cell numbers were counted after 13-14 days.

The colony formation assays were conducted as described previously (20). 48 hours after transfection equal numbers of cells were plated in triplicate into 6-well dishes containing medium with G418 (Sigma) at the same concentrations as the cell proliferation assay. Medium was changed every 3-4 days and colonies were counted after 13-14 days by washing with PBS, fixing with methanol and staining with Giemsa.

### Western Blot

Western blots were performed as previously described (18). Specifically, nuclear protein extracts were separated by SDS/polyacrylamide gel electrophoresis and transferred onto a nitrocellulose membrane. Membranes were hybridized with antibodies against EZH2 (Cell Signaling Technology). Total RNA (5 mg) was used for reverse transcription. After incubation with DNase I (Invitrogen) to eliminate DNA contamination, Superscript III (Invitrogen) and random hexamers (Promega, Madison, Wis.) were added for first strand cDNA synthesis. Then PCR was performed with primers specific for EZH2 (forward ATTTTTGTGAAAAGTTTTGTCAATGTAGTTCAGAG reverse TCACACTCTCGGACAGCCAG probe FAM-CAACACCAAGCAGTGCCCGTGCT-BHQ).

### Luciferase Assay

Reporter vectors were created by cloning the wild type or mutated 3′UTR of EZH2 into the XbaI site of the pGL3-control vector (Promega). Firefly and renilla luciferase activity was analyzed using the Dual. Luciferase Reporter assay system (Promega) as previously described (18). Specifically, luciferase constructs were made by ligating oligonucleotides containing the wild-type or mutant target site of the 3′UTR of EZH2 into the XbaI site of the pGL3-control vector (Promega). LD419 or HeLa cells were transfected with 0.4 mg of firefly luciferase reporter vector containing a wild-type ormutant target site and 0.02 mg of the control vector containing Renilla luciferase, pRL-CMV (Promega), using Lipofectamine 2000 (Invitrogen) in 24-well plates. Luciferase assays were performed 48 hr after transfection using the Dual Luciferase Reporter Assay System (Promega). Firefly luciferase activity was normalized to Renilla luciferase activity.

mRNA Microarray

UM-UC-3 cells were transfected with pre-miR-101, control precursors,

siRNA to EZH2, and control siRNA in triplicate. Total RNA was prepared 72 hours after transient transfection and the Illumina human 6 v 2 array was used for gene expression analysis. The Norris Cancer Center CORE facility performed the amplification and hybridization according to the manufacturer's protocol (Illumina).
microRNA Prediction Tools

To identify miRNAs targeting EZH2, the inventors integrated the output results of multiple prediction programs; TargetScan [http://www.targetscan.org/] (39), PicTar [http://pictar.org/] (40), miRanda [http://www.microrna.org/microrna/](41), and miRInspector [http://mirna.imbb.forth.gr/microinspector/](6). Each program was selected to leverage the various strengths for predicting miRNA targets in the areas of sequence alignment, thermodynamics, and comparative genomics. TargetScan requires a perfect seed, a sub-region of alignment between the miRNA and mRNA, while incorporating traditional RNA folding calculations and conservation of the binding site across vertebrates. PicTar, while preferring a perfect seed match, tolerates imperfect seed matches when they simultaneously adhere to heuristically defined thermodynamic requirements. miRanda employs a dynamic programming algorithm to establish miRNA:mRNA sequence alignment in addition to thermodynamics and conservation across multiple species. Lastly, microInspector identifies possible binding sites within an mRNA sequence relying heavily on free energy values at a binding site. The inventors developed a Perl script that imported the various output formats from each of the target prediction programs and subsequently integrated the results to detect common overlaps. For instance where all programs report an miRNA:mRNA interaction, the candidate miRNAs are sorted based on the predefined rankings from each respective program. Additionally, the inventors exported the number of predicted binding sites for miR-101 in the EZH2 3′UTR.

### Cell Lines

Breast cancer cell line SKBr3 were grown in RPMI 1640 (Invitrogen, Carlsbad, Calif.) with 10% FBS (Invitrogen) in 5% CO2 cell culture incubator, and prostate cancer cell line DU145 were grown in MEM with 10% FBS in 5% CO2 cell culture incubator. Immortalized breast cell lines HME and H16N2 were grown in F-12 Nutrient Mixture with 5 ug/ml Insulin (Sigma,), 1 ug/ml Hydrocortisone (Sigma), 10 ng/ml EGF (Invitrogen), 5 mM Ethanolamine (Sigma) 5 ug/ml Transferrin (Sigma), 10 nM Triiodo Thyronine (Sigma), 50 nM Sodium Selenite (Sigma), 10 mM HEPES (Invitrogen) and 50 unit/ml Penstrep (Invitrogen), 10% CO2. For mir-101 overexpression, pMIF-cGFP-Zeo construct expressing mir-101 was obtained from System Biosciences (Mountain View, Calif.). Lentiviruses were generated by the University of Michigan Vector Core. Prostate cancer cell line DU145 and breast cancer cell line SKBR3 were infected with lentiviruses expressing mir-101 or vector only, and stable cell lines were generated by selection with 300 ug/ml zeocin (Invitrogen, Carlsbad, Calif.). To generate stable EZH2 knockdown, shRNA lentiviral particles for EZH2 gene silencing and control vector were obtained from Sigma-Aldrich (St. Louis, Mo.). Prostate cancer cell line DU145 was infected with EZH2 shRNA lentivirus and a stable cell line was generated by selection with 1 ug/ml Puromycin (Sigma-Aldrich, St. Louis, Mo.).

### Tissues

In this study, the inventors utilized tissues from clinically localized prostate cancer patients, who underwent radical prostatectomy as a primary therapy between 2004-2006 at the University of Michigan Hospital, androgen-independent metastatic prostate cancer patients from a rapid autopsy program described previously (4,42), and patients with invasive carcinomas of the breast. The detailed clinical and pathological data were maintained on a secure relational database. This study was approved by the Institutional Review Board at the University of Michigan Medical School. Both radical prostatectomy series and the rapid autopsy program were part of the University of Michigan Prostate Cancer Specialized Program of Research Excellence Tissue Core. Breast cancer tissues were collected with IRB approval from the University of Singapore/National University Hospital, Singapore (NUS/NUH). The gastric cancer samples were collected with IRB approval from the National Cancer Center, Singapore.

microRNA Transfection, AntagomiR Transfection, and Small RNA Interference

Knockdown of EZH2 was accomplished by RNA interference using siRNA duplex (Dharmacon, Lafayette, Colo.) as previously described (43). Precursors of respective microRNAs and negative controls used in this study were purchased from Ambion (Austin, Tex.). AntagomiR-101 and negative control antagomiRs were purchased from Dharmacon. Transfections were performed with oligofectamine or lipofectamine (Invitrogen) depending on the cell line used.

### miR Reporter Luciferase Assays

The 3′UTR (untranslated region) or the antisense sequence of the 3′UTR of EZH2 as well as mutant 3′UTR of EZH2 were cloned into the pMIR-REPORT™ miRNA Expression Reporter Vector (Ambion). SKBr3 cells were transfected with pre-miR-101 or controls and then co-transfected with 3′-UTR-luc or mutant 3′UTR-luc, as well as pRL-TK vector as internal control for luciferase activity. Post 48 hours of incubation, the cells were lysed and luciferase assays conducted using the dual luciferase assay system (Promega, Madison, Wis.). Each experiment was performed in triplicate.

### Quantitative Real-Time PCR Assays

Total RNA was isolated from SKBr3 and DU145 cells that were transfected either with pre-miR-101, or control precursors (Qiagen). Quantitative PCR (QPCR) was performed using SYBR Green dye on an Applied Biosystems 7300 Real Time PCR system (Applied Biosystems, Foster City, Calif.) as described (45). Briefly, 1 μg of total RNA was reverse transcribed into cDNA using SuperScript III (Invitrogen, Carlsbad, Calif.) in the presence of random hexamers and oligo dT primers (Invitrogen). All reactions were performed in triplicate with SYBR Green Master Mix (Applied Biosystems) plus 25 ng of both the forward and reverse primer according to the manufacturer's recommended thermocycling conditions, and then subjected to melt curve analysis. Threshold levels for each experiment were set during the exponential phase of the QPCR reaction using Sequence Detection Software version 1.2.2 (Applied Biosystems). The DNA in each sample was quantified by interpolation of its threshold cycle (Ct) value from a standard curve of Ct values, which were created from a serially diluted cDNA mixture of all samples. The calculated quantity of the target gene for each sample was divided by the average sample quantity of the housekeeping genes, glyceraldehyde-3-phosphate dehydrogenase (GAPDH) to obtain the relative gene expression. All oligonucleotide primers were synthesized by Integrated DNA Technologies (Coralville, Iowa). The primer sequences for the transcript analyzed are provided in Table 1. For microRNA quantitative PCR, total RNA including small RNA was isolated from prostate tissues, SKBr3 and DU145 cells that were transfected either with pre-hsa-miR-101 (precursor human miRNA-101), or control precursors. Total RNA was used at 10 ng/ul. For RT, master mix were prepared using 0.15 ul 100 mM dNTPs, 1.00 ul MultiScrible Reverse Transcriptase (50 U/ul), 1.50 ul 10× Reverse Transcription Buffer, 0.188 ul RNase Inhibitor (20 U/ul) and 4.192 ul Nuclease-free water. Each 15 ul RT reaction mix contained, 7 ul of master mix, 5 ul of RNA samples (10 ng/ul) and 3 ul 5× specific RT primer. Thermal cycler was programmed as follows: 16 degrees for 30 minutes, 42 degrees for 30 minutes and 85 degrees for 5 minutes. Each PCR reaction mix contained 10 ul of Taqman 2× Universal PCR Master Mix (No AmpErase UNG), 6.67 ul Nuclease-free water, 1 ul 20× specific PCR primer and 1.33 ul RT product. Thermal cycler was programmed as follows: 95 degrees for 10 minutes, 40 cycles of 95 degrees for 15 seconds and 60 degrees for 60 seconds. Using the comparative CT method, the inventors used endogenous control (RNU6B) to normalize the expression levels of target micro-RNA by correcting differences in the amount of RNA loaded into qPCR reactions.

### Genomic PCR Assays

Genomic DNA from benign (n=15), localized prostate cancer (n=16) and metastatic (n=33) prostate cancer tissues were isolated. DNA from benign (n=7), tumor (n=29) and metastatic (n=1, three different sites) from breast cancer cases were also isolated. For genomic analyses of the miR-101 loci, the 2̂-ΔΔCt method was adapted using SyBr green based quantitative PCR (qPCR) (44, 45). Briefly, 100 ng 25 ng of gDNA was used as template to amplify the miR-101-1, miR-101-2 and miR-217 encompassing loci. Since miR-217 levels did not show significant correlation with EZH2 transcript levels, miR-217 was used as the reference for relative quantification. The assay was validated using the methods described (44, 45). For unification of data and to avoid inter-assay differences Ct values for the reference gene (miR-217) were always estimated simultaneously with miR-101-1 or miR-101-2. A representative benign tissue sample was used in every assay as a calibrator sample to which every sample was compared, to obtain a relative quantitation (RQ) value. To calibrate the extent of loss in the miR-101 loci the inventors determined the relative levels of 9 different genomic regions on X-chromosome (three regions in phosphoglycerate kinase 1 (PGK1) gene, and six X-chromosome specific miRNAs- miR-424, miR-503, miR-766, miR-448, miR-222 and miR-221) in the genomic DNA from a normal male sample (1×) as compared to a normal female sample (2×) genomic DNA (Promega) that are located only on the X chromosome. RQ values for these regions in male genomic DNA were assessed using a non-Xchromosome gene Tata Binding Protein (TBP) gene as the reference gene (45). An RQ value of 0.7 and below was considered as loss of at least one copy of the genomic loci (Table 2), similar to earlier reports (44, 45). Accordingly, samples showing values lower than 0.7 were considered to have a hemizygous loss and those below 0.3 were considered to exhibit a homozygous loss. For the loss of heterozygosity (LOH) analysis, 9 cancer samples showing miR-101-1 deletion were identified and normal (non prostatic) tissues from the same cases were obtained. Genomic qPCR analysis was carried out in these as described above and RQ values obtained were compared to those obtained from the matched cancer cases. Primer sequences used for genomic PCR assays are given in Table 3.

### Immunoblot Analyses

The breast cancer cell lines SKBr3 and prostate cancer cell DU145 were transfected with pre-miR-101 or controls. The breast cell lines H16N2 and HME were transfected with antagomiR-101 or negative controls. Post 72 hours transfection, cells were homogenized in NP40 lysis buffer (50 mM Tris-HCl, 1% NP40, pH 7.4, Sigma, St. Louis, Mo.), and complete proteinase inhibitor mixture (Roche, Indianapolis, Ind.). Ten micrograms of each protein extract were boiled in sample buffer, separated by SDS-PAGE, and transferred onto Polyvinylidene Difluoride membrane (GE Healthcare, Piscataway, N.J.). The membrane was incubated for one hour in blocking buffer [Tris-buffered saline, 0.1% Tween (TBS-T), 5% nonfat dry milk] and incubated overnight at 4° C. with the following: anti-EZH2 mouse monoclonal (1:1000 in blocking buffer, ED Biosciences Cat #612667, San Jose, Calif.), anti-EED rabbit polyclonal (1:1000, Santa Cruz Biotech, Cat #: sc-28701, Santa Cruz, Calif.), anti-SUZ12 mouse monoclonal (1:1000, Upstate, Cat #; 04-046, Charlottesville, Va.), and anti-N-myc rabbit polyclonal antibodies (1:1000, Cell Signaling Tech, Cat #: 9405, Danvers, Mass.), anti-ARID1A mouse monoclonal antibody (1:1000, Abcam, Cat #: ab50878, Cambridge, Mass.), anti-FBN2 rabbit polyclonal antibody (1:1000, Abeam, Cat #: ab21619), anti-c-Fos mouse monoclonal antibody (1:1000, ED Biosciences, Cat #: 554156), antitrimethyl-H3K27 rabbit polyclonal (1:2000, Upstate, Cat #: 07-449), anti-monomethyl-Histone H3 (Lys27) (1:1000 upstate Cat #: 07-448), anti-acetyl-Histone H3 (K27) (upstate Cat #: 07-360) and antitotal Histone H3 rabbit polyclonal (1:5000, Cell Signaling, Cat #: 9715) and anti-GAPDH mouse monoclonal antibody (1:10000, Abeam, Cat #: ab9482). Following a wash with TBS-T, the blot was incubated with horseradish peroxidase-conjugated secondary antibody and the signals visualized by enhanced chemiluminescence system as described by the manufacturer (GE Healthcare).

### Cell Proliferation Assay

Cells were plated in 24-well plates at desired cell concentration and transfected with precursor microRNA or controls. Cell counts were estimated by trypsinizing cells and analysis by Coulter counter (Beckman Coulter, Fullerton, Calif.) at the indicated time points in triplicate.

### Cell Migration Assay Using Wound Healing Assay

DU145 lenti-vector and miR-101 overexpressing, and sh-vector and EZH2 knockdown stable cells were grown to confluence. An artificial wound was created using a 1 ml pipette tip on confluent cell monolayer. To visualize migrated cells and wound healing, cell images were taken at 0, 24, 48 and 72 hrs.

### Basement Membrane Matrix Invasion Assays

For invasion assays, the breast cell lines H16N2 and HME were transfected with antagomiR-101 or negative controls. Invasive breast cancer cell SKBr3 and prostate cancer cell DU145 were transfected with pre-miR-101 or controls. Forty-eight hours post-transfection, cells were seeded onto the basement membrane matrix (EC matrix, Chemicon, Temecula, Calif.) present in the insert of a 24 well culture plate. Fetal bovine serum was added to the lower chamber as a chemoattractant. After 48 hours, the noninvading cells and EC matrix were gently removed with a cotton swab. Invasive cells located on the lower side of the chamber were stained with crystal violet, air dried and photographed. For colorimetric assays, the inserts were treated with 150 μl of 10% acetic acid and the absorbance measured at 560 nm using a spectrophotometer (GE Healthcare).

### Soft Agar Colony Formation Assays

A. 50 μL base layer of agar (0.6% Agar in DMEM with 10% FBS) was allowed to solidify in a 96-well flat-bottom plate prior to the addition of a 75 μL wild type DU145, DU145 miR-101 clones or vector transfected DU145 cell suspension containing 4,000 cells in 0.4% Agar in DMEM with 10% FBS. The cell containing layer was then solidified at 4 C for 15 minutes prior to the addition of 100 μL of MEM with 5% FBS. Colonies were allowed to grow for 21 days before imaging under a light microscope.

### Prostate Tumor Xenograft Model

All procedures involving mice were approved by the University Committee on Use and Care of Animals (UCUCA) at the University of Michigan and conform to their relevant regulatory standards. Five-week old male nude athymic BALB/c nu/nu mice (Charles River Laboratory, Wilmington, Mass.) were used for examining the tumorigenicity. To evaluate the role of miR-101 overexpression in tumor formation, the DU145 stable cells overexpressing miR-101 or vector control cells were propagated and 5×106 cells were inoculated subcutaneously into the dorsal flank of ten mice (n=5 per group). Tumor size was measured every week, and tumor volumes were estimated using the formula (n/6) (L×W2), where L=length of tumor and W=width

### Chromatin Immunoprecipitation (ChIP) Assays

The effect of miR-101 over-expression on trimethyl H3 (Lys-27) status of EZH2 targets was determined by Chromatin Immunoprecipitation (ChIP) assay. The ChIP assay was carried out with antibodies against trimethyl H3 (Lys-27) (Mouse monoclonal from Abeam, Cat: Ab6002-100). The assay was performed using the EZ-Magna ChIP kit (Millipore) according to the manufacturer's protocol. Briefly, 2×106 cells were used for each immunoprecipitation. The cells were cross-linked for 10 minutes by addition of formaldehyde to a final concentration of 1%. The cross-linking was stopped by 1/20V of 2.5M glycine. This was followed by cell lysis and sonication, resulting in an average fragment size of 500 bp. Antibody incubations were carried out over-night at 4° C. Reversal of cross-linking was carried out at 65° C. for 3 hours, followed by DNA isolation. The purified DNA was analyzed by quantitative PCR to determine fold enrichment relative to input DNA. The primer sequences for the promoters analyzed are provided in Table 4.

### Gene Expression Profiling

Expression profiling was performed using the Agilent Whole Human Genome Oligo Microarray (Santa Clara, Calif.) according to the manufacturer's protocol. SKBr3 cells were transfected with pre-miR-101 or negative control for precursor microRNA. Over- and under-expressed signatures were generated by filtering to include only features with significant differential expression (Log ratio, P<0.01) in all hybridizations and two-fold average over- or under-expression (Log ratio) after correction for the dye flip. To ensure that the inventors were comparing robust gene expression alterations, the inventors analyzed biological replicates and used only the probes showing expression changes in both replicates.

### Array Comparative Genomic Hybridization

Comparative genomic hybridization analysis for prostate, breast and gastric cancers were carried using oligonucleotide based comparative genomic hybridization array (Hg17 genome build) (Agilent Technologies, USA) according the manufacture's instructions. Competitive hybridization of differentially labeled tumor and reference DNA to oligonucleotide printed in an array format and analysis of fluorescent intensity for each probe will detect the copy number changes in the tumor sample relative to normal reference genome. The inventors analyzed copy number changes for miR-101-1 (1p31.3) and miR-101-2 (9p24.1) regions with a change in copy number level of at least one copy (log ratio±0.5) for losses involving more than one probe representing each genomic interval as detected by the aberration detection method (ADM-2) in CGH analytics software 3.5 Agilent Technologies) algorithm.

Analysis of Publicly Available Array CGH/SNP Datasets for miR-101 Copy Number Analysis

To examine the mir-101 loss status in multiple cancers, the inventors collected the public array CGH/SNP datasets from Gene Expression Omnibus (http://www.ncbi.nih.gov/geo) and Cancer Bioinformatics Grid (https://cabig.nci.nih.gov/). Acute lymphoblastic leukemia (46), glioblastoma (Data from TOGA) and lung cancer (3) studies were analysed. The sample information was manually curated and classified into cancer (primary plus metastasis), metastasis and normal samples. For the Affymetrix SNP arrays, model-based expression was performed to summarize signal intensities for each probe set, using the perfect-match/mismatch (PM/MM) model. For copy number inference, raw copy number was calculated by comparing the signal intensity of each SNP probe set for each tumor sample against a diploid reference set of samples. All of the resulting DNA copy number ratios were transformed by log 2. In the two-channel array CGH datasets, the differential ratio between the processed testing channel signal and processed reference channel signal were calculated and transformed by log 2, which reflects the DNA copy number difference between the testing channel and reference channel. In the normalization step, the log ratios were transformed into a normal distribution with a mean of 0 under the null model assumption. The data were segmented by circular binary segmentation (CBS) algorithm developed by Olshen et al, (3) a method for identifying all genomic change points where the mean log ratio score changes between intervals. The threshold for deletion was modified from the report by Mullighan et al, (46). Cutoffs of 0.9 and 0.3 were used to identify hemizygous and homozygous deletions, respectively. The probe closest to the selected gene was used to represent the DNA copy number status of this gene, with a maximal distance of 10 kb.

### Statistical Analyses

All gene expression and relative quantification data were analyzed on the log (base 2) scale. Comparisons between gene expression values across sample classes were made using two-sample Student's t-test. The significance of associations between EZH2 and miR-101 expression values was judged via a test statistic based on Pearson's product moment correlation coefficient. Associations between binary variables (loss at the two miR-101 loci, and overlap of gene sets) were explored using Fisher's exact test. The relationship between EZH2 overexpression and miR-101 loss was evaluated using a test statistic calculated as the minimum observed value of EZH2 expression in the set of samples exhibiting miR-101 loss at either locus. The null permutation distribution of this statistic was derived by randomly permuting miR-101 loss status within the set of samples; N=10000 permutations were used. The significance of the separation between miR-101 and vector trajectories in the mouse xenograft model was evaluated via a linear mixed model that incorporated a random intercept for each mouse and used square-root transformed tumor volume measurements as dependent variable. Wald tests were used to assess the statistical significance of observed differences between growth rates in the two groups of mice. All statistical tests were two-sided and constructed at the α=0.05 significance level except for the above described permutation test, which was one-sided and conducted at the α=0.025 significance level. Statistical analyses were performed using R, version 2.7.0 (http://www.r-project.org).

### Results and Discussion

Differential miRNA Expression in TCCs

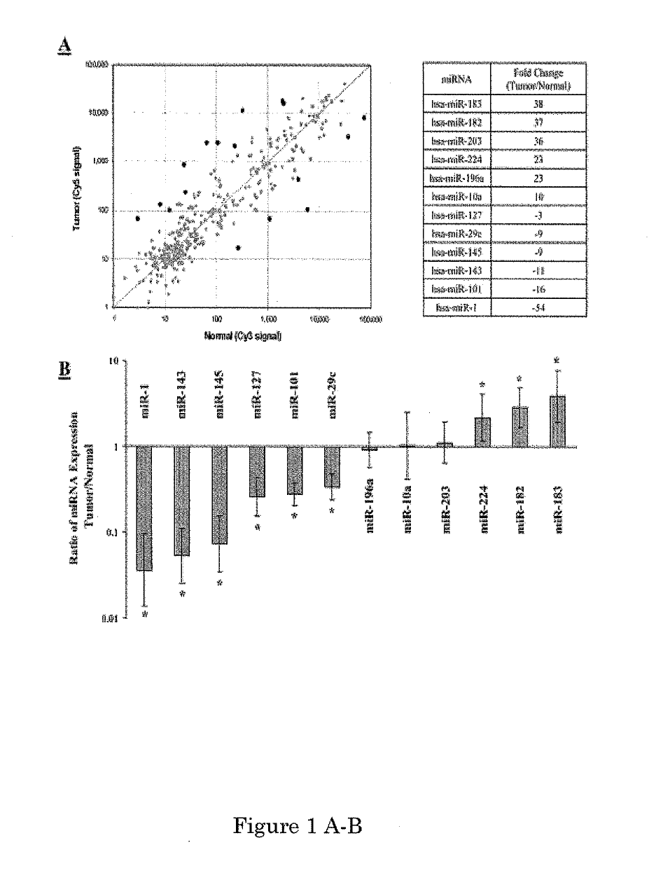

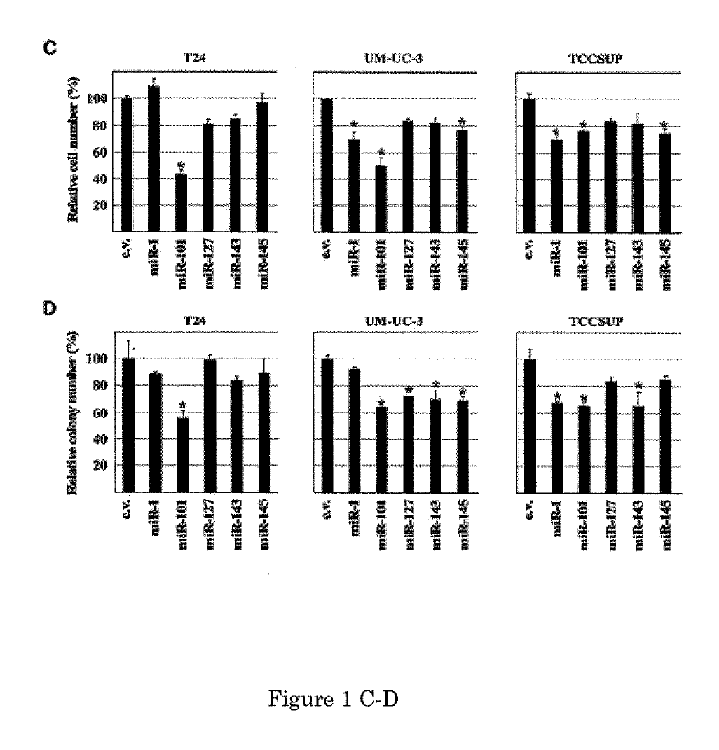

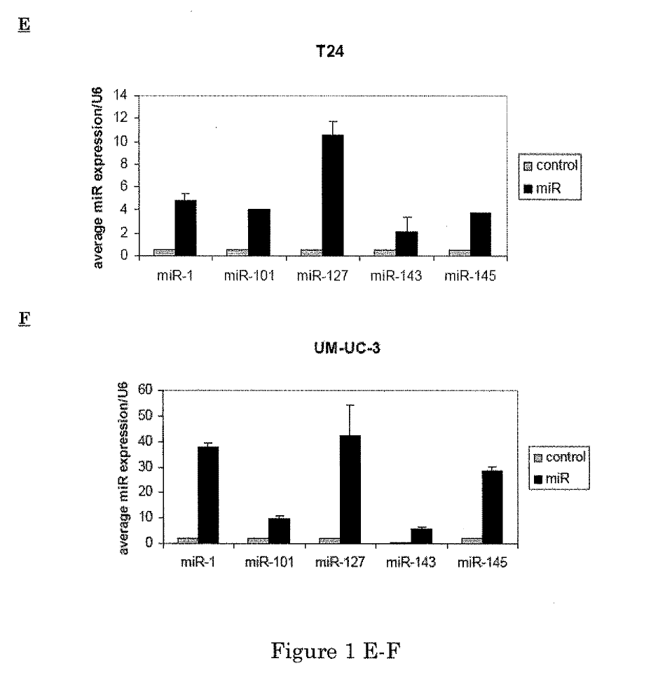

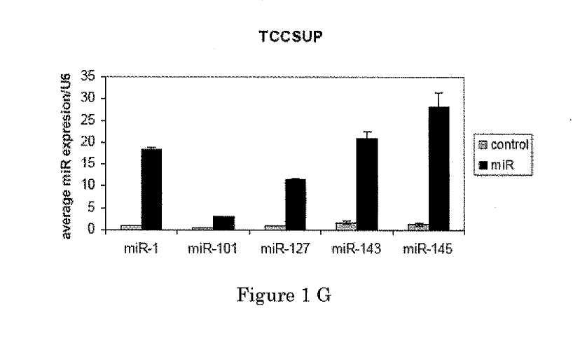

The inventors used a miRNA microarray to examine differentially expressed miRNAs in a pool of 9 TCCs and a pool of the corresponding normal samples. miR-1, miR-101, miR-143, miR-145 and miR-29c were the most downregulated transcripts in the tumors and miR-182, miR-183, miR-203, miR-224 and miR-196a were the most upregulated (FIG. 1A). miR-127 was included in the table because our previous work revealed it is downregulated in human cancers (18).

The inventors conducted RT-qPCR for 12 differentially expressed miRNAs on 28 additional TOO patients. miR-1, miR-101, miR-143, miR-145, miR-29c, and miR-127 were significantly downregulated in the tumors, while miR-224, miR182, and miR-183 were significantly upregulated in the tumors (FIG. 1B). miR-196a, miR-10a and miR-203 showed no significant differences. The miRNA microarray analysis and RT-qPCR results showed that there is severe and consistent miRNA misexpression in TOO and our miRNA panel likely constitutes a TOO miRNA signature. The miRNA that was most downregulated in TOO, miR-1, and miR-29c were down-regulated in hepatocellular carcinoma and lung cancer, respectively (21, 22). Furthermore, the study supports the link between downregulation of miR-143 and miR-145 and cancer, which was previously shown in various malignancies (23, 24).

Restored Expression of miRNAs in TCC Cell Lines Reveals Putative Tumor Suppressors

The inventors used RT-qPCR to analyze miRNA expression in 10 TCC cell lines. miR-1, miR-101, miR-127, miR-143, and miR-145 were expressed at low levels in all cell lines, indicating these miRNAs are promising tumor suppressor candidates. After creating miRNA expression vectors, T24, TCCSUP and UM-UC-3 cells were transfected and the enhanced miRNA expression from each vector was confirmed by RT-qPCR (FIG. 1 E, F, and G).

Proliferation assays were clone by stably transfecting cells with the miRNA expression vectors to determine the functional effects of miRNA misexpression. The results showed a strong inhibition of cell proliferation by

miR-101. In T24 cells, miR-101 suppressed proliferation by 57% (FIG. 1C). Proliferation was significantly inhibited by miR-101, miR-1, and miR-145 in UM-UC-3 and TCCSUP cells (FIG. 1C). These results indicate that miR-101 is the most potent growth suppressor, although miR-1 and miR-145 also significantly inhibited cell proliferation.

The inventors determined the effect of restored miRNA expression on colony formation and again, the results varied depending on the cell line and miR-101 was the most potent suppressor of colony formation. In T24 cells, miR-101 suppressed colony formation by 45% (FIG. 1D). miR-101, miR-127, miR-143, and miR-145 significantly reduced colonies in UM-UC-3 cells, while in TCCSUP cells, miR-101, miR-1, and miR-143 showed significant suppression (FIG. 1D). Clearly, restored expression of miR-101 potently suppresses colony formation in these cell lines while other miRNAs also suppress colony formation, although less substantially and consistently. The cell proliferation and colony formation assays indicate that miR-101 is a putative tumor suppressor and may be a therapeutic target for cancer.

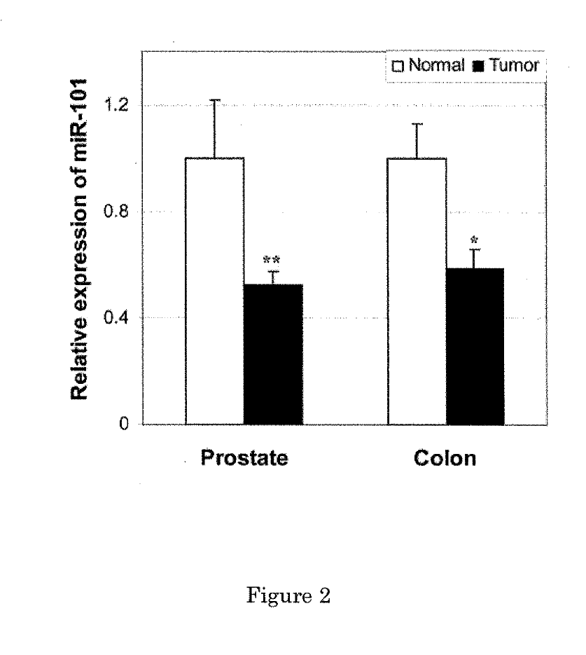

Because previously published reports showed that miR-101 was downregulated in lung cancer and breast cancer (23, 24), miR-101 expression was examined by RT-qPCR in colon and prostate tumors as well as the corresponding matched normal tissues. The inventors found that miR-101 was downregulated in bath colon tumors (6/10) and prostate tumors (4/7) (FIG. 2). The results and previously published data indicate that miR-101 is downregulated in the 5 most frequently diagnosed cancers in the U.S. (NCI), indicating that miR-101 might be part of a solid tumor signature.

There are a plethora of mechanisms that could lead to decreased miRNA expression in cancer including copy number alterations (17), epigenetic silencing (18) and trans-acting factors (16). There are two copies of miR-101 with miR-101-1 located at chromosome 1p31 and miR-101-2 located at chromosome 9p24. The two precursors have different sequences but the mature forms are identical. The inventors found that miR-101 silencing in TCC cell lines was probably not due to epigenetic phenomena because treatment with the DNA demethylating agent 5-aza-2′-deoxycytidine and the histone deacetylase inhibitor 4-phenylbutyric acid did not induce expression of miR-101 (data not shown). Intriguingly, loss of heterozygosity (LOH) at chromosome 1p occurs in many different solid tumors and is negatively associated with survival (25), while LOH at chromosome 9p also commonly occurs in cancer, particularly TCC (26). This suggests that DNA

copy number may regulate miR-101 expression, although a trans-acting mechanism should be investigated in the future.
miR-101 Represses the Polycomb Group Protein EZH2

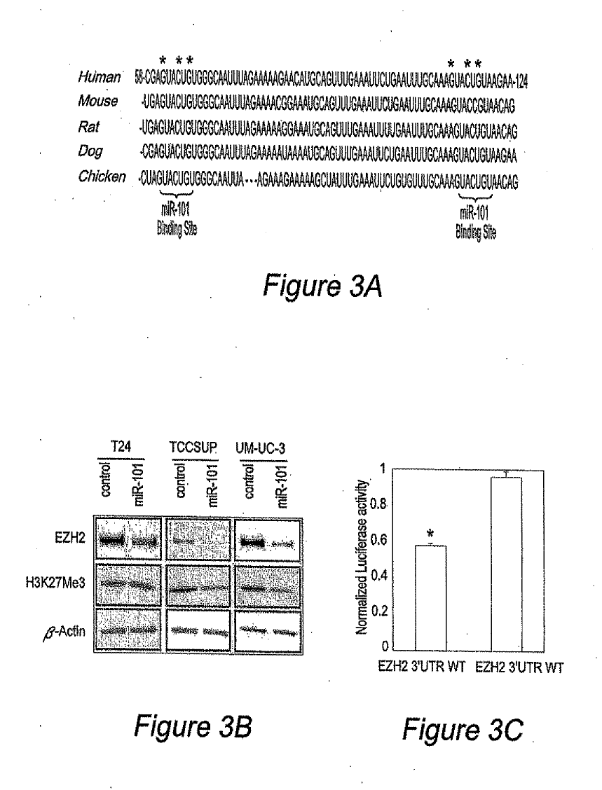

The inventors used the prediction algorithm TargetScan (www.targetscan.org) to identify targets of miR-101 and found the histone methyltransferase EZH2 had a very high score, a highly conserved sequence, and two predicted sequence matches to the miR-101 seed (FIG. 3A). The inventors transiently transfected miR-101 precursors into T24, TCCSUP, and UM-UC-3 cells and visualized EZH2 levels by Western blot. The transient transfections of precursor miRNAs were designed to determine miRNA targets whereas the stable transfections of miRNA expression vectors examined the long term effects on cell growth (FIG. 1). In TCCSUP and UM-UC-3 cells, EZH2 levels decreased by 86% and 91%, respectively, while HBK27me3 decreased by 58% in both cell lines (FIG. 313). In T24 cells there was a 52% decrease in EZH2 levels and the H3K27me3 levels remained the same which is likely due to lower transfection efficiencies that yielded 50% less mature miR-101 levels when compared to UM-UC-3 cells (data not shown). These results indicate that miR-101 can repress EZH2 in TCC cell lines (FIG. 3B).

EZH2 is a Direct Target of miR-101

To confirm that EZH2 is a direct target of miR-101, the inventors created luciferase reporter vectors by cloning either the wild type or a mutated portion of the 3′UTR of EZH2 into the 3′UTR of the pGL3-control vector. The mutated 3′UTR had three bases changed, from GUACUGU to CUAGUCU, at each of the two putative miR-101 binding sites (FIG. 3A). The inventors transfected these vectors with pre-miR-101 into UM-UC-3 cells and the lysates were analyzed 24 h later. The results showed a 42% decrease in luciferase activity for the wild type 3′UTR of EZH2, while the mutated 3′UTR showed no repression when compared to the empty reporter as was also found previously (27) (FIG. 30). Therefore, miR-101 represses EZH2 by binding to the 3′UTR of EZH2 in a direct and sequence specific manner.

siRNA to EZH2 Shows Phenotypic Overlap with the Restoration of miR-101 Expression in UM-UC-3 Cells

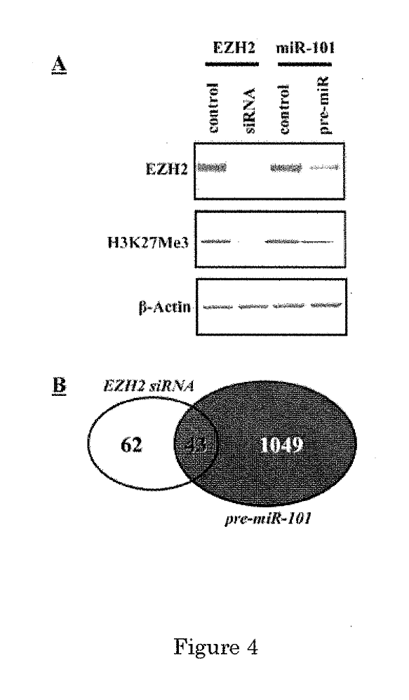

After confirming that miR-101 decreases H3K27me3 by targeting EZH2, the inventors examined if there was a phenotypic overlap between pre-miR-101 transfection and knockdown of EZH2 by siRNA in UM-UC-3 cells (FIG. 4A). Expression microarrays were conducted on UM-UC-3 cells transfected with pre-miR-101 or siRNA to EZH2. The inventors identified a significant overlap of 43 upregulated genes, which suggests that restoring miR-101 expression in cancer cells re-expresses a subset of genes that are repressed by EZH2 (FIG. 48). The siRNA to EZH2 caused a more efficient knockdown of EZH2 and H3K27me3 than the pre-miR-101 transfection (FIG. 4A). The extra decrease in EZH2 and H3K27me3 from the siRNA to EZH2 may explain why there were 62 upregulated genes that did not overlap with the genes upregulated by pre-miR-101 transfection (FIG. 4B). In contrast, the inventors expect the pre-miR-101 transfection to have much wider effects on gene expression than the siRNA to EZH2 because miRNAs likely repress several targets that influence the transcriptome.

The molecular similarities between cancer and stem cells are becoming

ever more apparent. For example, EZH2 is critical for the maintenance of proliferation and pluripotency in stem cells (1). Global levels of Ezh2 and H3K27me3 decrease when mouse embryonic stem (ES) cells differentiate and PRC2 target genes specifically lose Ezh2 binding and H3K27me3 enrichment (28). In addition, two groups found that miR-101 was upregulated after differentiation of human ES cell lines (29, 30). These reports present a strong association between miR-101 activation and EZH2 repression upon differentiation of stem cells, suggesting that miR-101 might be part of the complex network, which includes PcG proteins, that determines developmental outcome. Therefore, in addition to its potential role in cancer, miR-101 might be involved in normal differentiation by directly repressing EZH2 and re-expressing cell fate regulating genes.

The role of miRNAs in controlling epigenetics is just emerging, ES cell

specific miRNAs control de novo DNA methylation during differentiation by targeting Rbl2 (31, 32). A recent report suggests that the downregulation of the miR-29 family in lung cancer could lead to the overexpression of DNA methyltransferases 3A and 3B and subsequent DNA hypermethylation and gene silencing (22). The results show another instance in which miRNA-mediated epigenetic mechanisms can be dysregulated in cancer with global consequences. Taken together, our results suggest that aberrant silencing of miR-101 may be a cause of the overexpression of EZH2 seen in cancer and that restoring miR-101 expression could hinder EZH2-mediated neoplastic progression.
shRNA to EZH2 Shows Phenotypic Overlap with the Restoration of miR-101 Expression in UM-UC-3 Cells

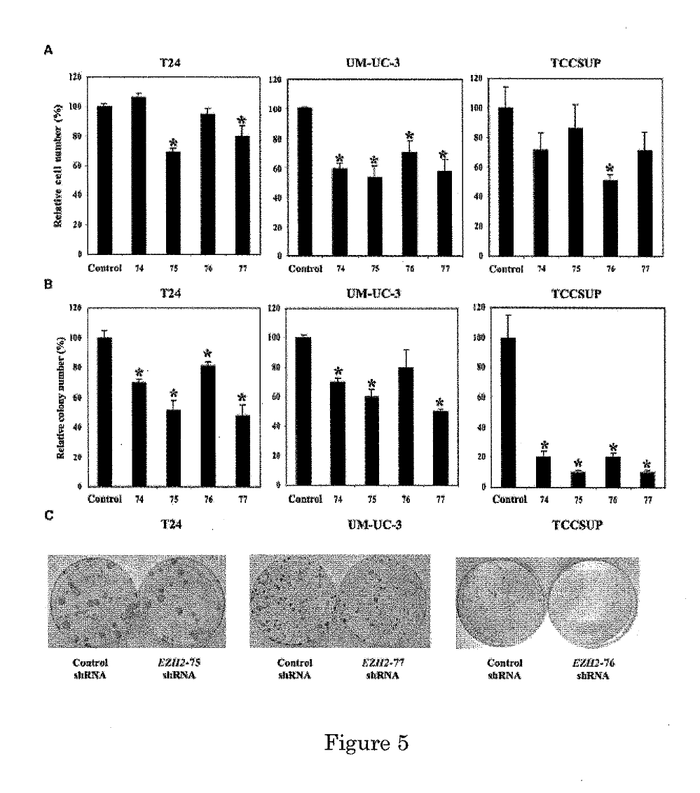

The inventors conducted cell proliferation assays and colony formation assays in three TCC cell lines using four expression vectors (clones 74, 75, 76, and 77) containing distinct shRNAs targeting EZH2. The results showed that two of four, one of four, and four of four shRNAs suppressed cell proliferation in T24, TCCSUP, and UM-UC-3 cells, respectively (FIG. 5A). The colony formation results were more striking as all four shRNAs suppressed colony formation in T24 and TCCSUP cells, whereas three of four shRNAs suppressed colony formation in UM-UC-3 cells (FIG. 5B and C).

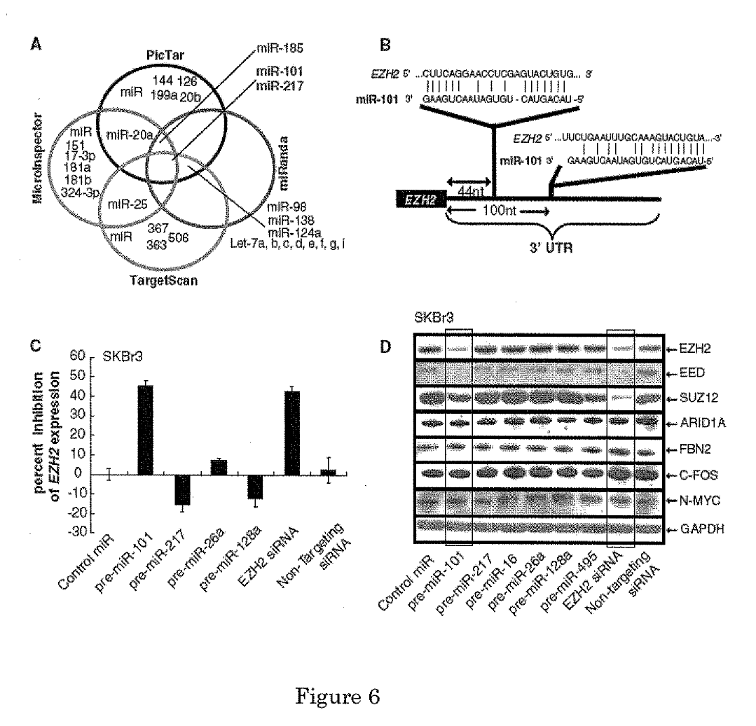

The results of the prediction software programs PicTar (47), TargetScan (27), miRanda (48), and miRInspector (49). Overall, only 29 miRNAs were found by any program to target EZH2, whereas only microRNA-101 (miR-101) and miR-217 were found by all four programs to be predicted to regulate EZH2 (FIG. 6A). Furthermore, PicTar, miRanda, and TargetScan predicted two miR-101-binding sites within the EZH2 3′ untranslated region (3′UTR) (FIG. 6B), whereas PicTar and TargetScan predicted two miR-217 binding sites within the EZH2 3′UTR. Of the 34 miRNAs predicted to regulate EZH2 by at least one program, only miR-101 had a strong negative association with prostate cancer progression from benign to localized disease to metastasis.

To examine whether miR-101 regulates the 3′UTR of EZH2, the inventors generated luciferase reporters encoding the normal, antisense, and mutated versions of the EZH2 3′UTR. Overexpression of miR-101, but not miR-217 or control miRNA, decreased the activity of the luciferase reporter encoding the 3′UTR of EZH2. Similarly, the antisense and mutant EZH2 3′UTR activities were not inhibited by miR-101. To explore whether the 3′UTR binding by miR-101 results in down-regulation of the EZH2 transcript, the inventors transfected SKBr3 breast cancer cells (which express high levels of endogenous EZH2) with precursors of miR-101, miR-217, and a control miRNA, as well as several other unrelated miRNAs. Quantitative reverse transcription polymerase chain reaction (RT-PCR) demonstrated a reduction in EZH2 transcript levels by miR-101 (FIG. 6C) but not miR-217 or other control miRs.

To determine whether miR-101 represses EZH2 protein expression, the inventors performed immunoblot analysis using an EZH2-specific antibody as well as antibodies to other PRC2 members, including EED and SUZ12 (FIG. 6D). In addition to miR-101, the inventors included other miRNAs that were predicted to regulate EZH2, including miR-217 and miR-26a. Control miR-101 was predicted by TargetScan to target the PRC1 component BMI-1. Only miR-101 and EZH2 small interfering RNA (siRNA) attenuated EZH2 protein expression. miR-101 overexpression also leads to repression of EZH2's tight binding partners in the PRC2 complex: EED and, to a lesser extent, SUZ12. These proteins are thought to form a coregulated functional complex, and altering the expression of one affects the expression of the others (4, 2, 50). In this particular case, upon further inspection of the 3′UTRs of the PRC2 components, miR-101 binding sites were found in EED but not in SUZ12. Because miRNAs are known to regulate multiple target genes, and in some cases hundreds of genes (51), the inventors used the prediction algorithm TargetScan to nominate targets of miR-101. In addition to EZH2 and EED, the inventors tested four predicted targets of miR-101 that have been implicated in cancer pathways, including n-Myc, c-Fos, AT-rich interactive domain 1A (also called SWI-like and ARID1A), and fibrillin 2 (FBN2). None of these putative miR-101 targets were affected by overexpression of miR-101 (FIG. 6D). To support the findings from our miR-101-overexpression experiments, the inventors employed antagomiR technology (52) to specifically inhibit miR-101 expression in benign immortalized breast epithelial cells. Two independent antagomiRs to miR-101 (i and ii) induced expression of EZH2 protein in benign breast epithelial cells.

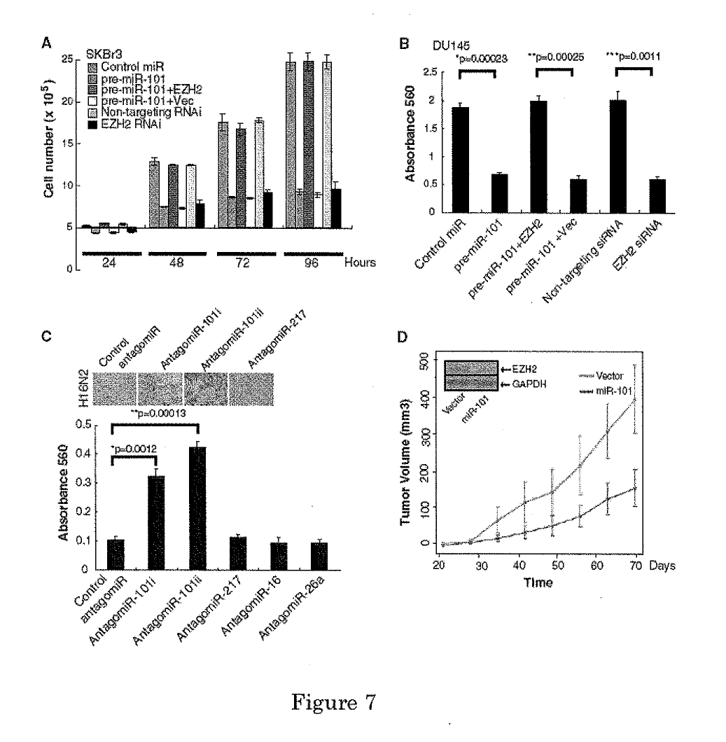

To determine whether miR-101 affects EZH2 and PRC2 function, the inventors evaluated cellular proliferation, a property known to be regulated by EZH2 (6, 4). miR-101 overexpression in SKBr3 and DU145 cells markedly attenuated cell proliferation (FIG. 7A). Overexpression of EZH2 (without an endogenous 3′UTR) rescued the inhibition of cell growth by miR-101, which suggests target specificity.

The inventors previously showed that upon overexpression, EZH2 can induce cell invasion in matrigel coated basement membrane invasion assays (3). Here the inventors show that miR-101 overexpression markedly inhibits the in vitro invasive potential of DU145 prostate-cancer cells (FIG. 7B) and SKBr3 breast cancer cells. Similarly, stable expression of miR-101 in DU145 cells showed a reduction in EZH2 expression and reduced invasion. Overexpression of EZH2 rescued the inhibition that was mediated by miR-101. Another in vitro readout for tumorigenic potential, increased cell migration, was also inhibited by miR-101. Because overexpression of miR-101 attenuates cancer invasion, inhibition of miR-101 should enhance this neoplastic phenotype. Two independent antagomiRs targeting miR-101 (i and ii) induced an invasive phenotype when transfected into benign immortalized breast epithelial cell lines H16N2 or HME (FIG. 7C).

To assess whether miR-101 inhibits anchorage independent growth, the inventors used a soft-agar assay. DU145 prostate cancer cells stably overexpressing miR-101 exhibited markedly reduced colony formation relative to the parental cells or vector controls. Furthermore, in vivo, DU145 cells expressing miR-101 grew significantly slower than the vector control xenografts (P=0.0001) (FIG. 7D), demonstrating that miR-101 has properties consistent with that of a tumor suppressor in these particular assays.

Because EZH2 and PRC2 regulate gene expression by trimethylating H3K27, the inventors believed that miR-101 overexpression would result in decreased overall H3K27 trimethylation in cancer cells. SKBr3 breast cancer and DU145 prostate cancer cells transfected with miR-101 or EZH2 siRNA for 7 days displayed a global decrease in trimethyl H3K27 The effect of miR-101 on H3K27 methylation was negated by overexpression of EZH2.

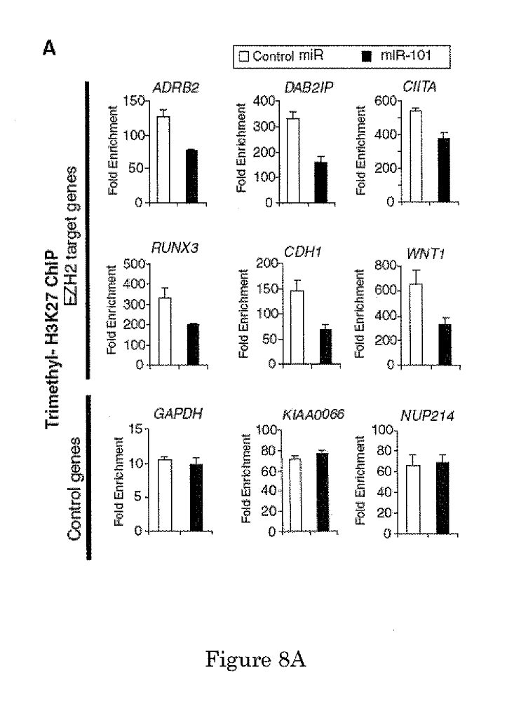

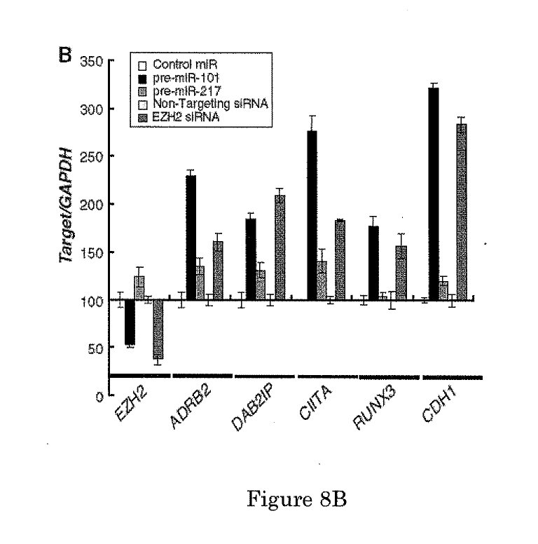

To test the level of promoter occupancy of the H3K27 histone mark, the inventors performed chromatin immunoprecipitation (ChIP) assays in cancer cells overexpressing miR-101. The inventors found significant reduction in the trimethyl H3K27 histone mark at the promoter of known PRC2 target genes such as ADRB2, DAB2IP, CIITA, and WNT1 in miR-101-overexpressing SKBr3 cells and EZH2 siRNA-treated cells (FIG. 8A). To determine whether the reduced promoter occupancy by H3K27 results in concomitant reduction of gene expression, the inventors performed quantitative RT-PCR on the PRC2 targets tested by ChIP assay. As expected, there was a significant increase in target gene expression in both miR-101- and EZH2 siRNA-treated cells (FIG. 8B). To further explore miR-101 regulation of EZH2 and its possible similarity with EZH2-specific RNA interference (RNAi), the inventors examined whether miR-101 overexpression and EZH2 knockdown generated similar gene expression profiles. To assess this, the inventors conducted gene-expression array analysis of SKBr3 cells transfected with either miR-101 or EZH2 siRNA duplexes. Genes that were overexpressed at the twofold threshold were significantly overlapping in both the miR-101- and EZH2 siRNA-transfected cells (P=6.08×10⁻¹⁷). Similarly, those genes that were repressed also had significant overlap (P=3.24×10²⁷).

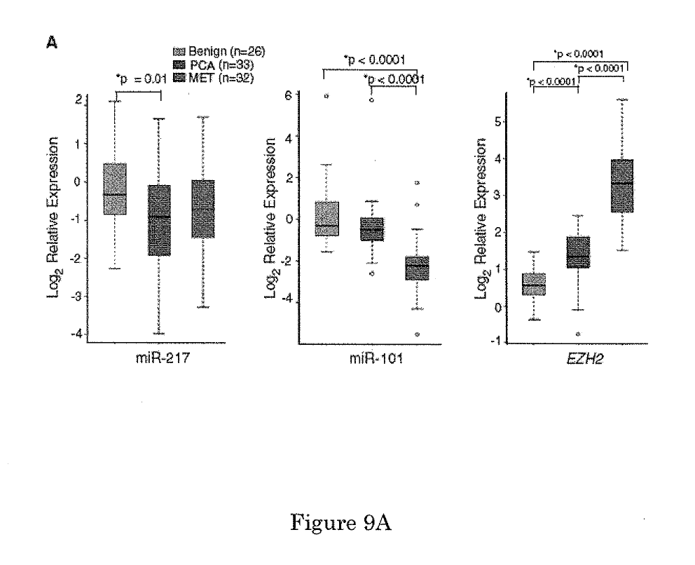

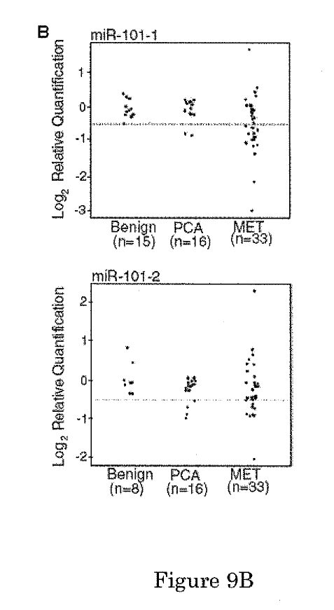

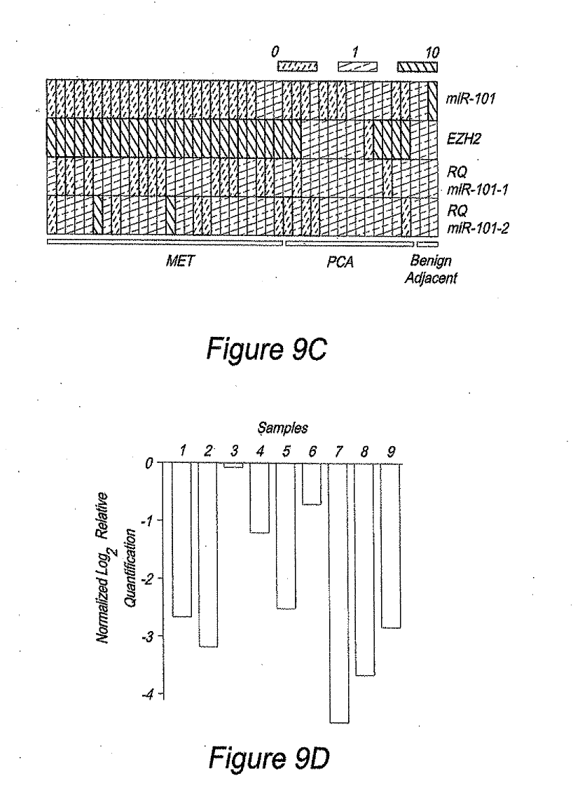

The inventors next investigated whether miR-101 expression inversely correlates with EZH2 levels in human tumors. A meta-analysis of a majority of the publicly available miRNA expression data sets suggested that miR-101 is significantly under expressed in prostate, breast, ovarian, lung, and colon cancers. Because EZH2 was initially found to be over expressed in a subset of aggressive clinically localized prostate cancers and almost universally elevated in metastatic disease (6), the inventors examined miR-101 in a similar context of prostate cancer progression by doing quantitative PCR analysis (FIG. 9A). As expected, metastatic prostate cancers expressed significantly higher levels of EZH2 as compared with those of clinically localized disease or benign adjacent prostate tissue (P<0.0001). Consistent with a functional connection between miR-101 and EZH2, miR-101 expression was significantly decreased in metastatic prostate cancer relative to that in clinically localized disease or benign adjacent prostate tissue (P<0.0001). miR-217, which like miR-101 was predicted to regulate EZH2, did not exhibit significant differences between metastatic disease and clinically localized prostate cancer or benign prostate tissue (P=0.35 and 0.13, respectively).

To investigate the mechanism for miR-101 transcript loss in prostate cancer progression, the inventors performed quantitative genomic PCR for miR-101. miR-101 has two genomic loci that are on chromosome 1 (miR-101-1) and chromosome 9 (miR-101-2). Based on genomic PCR, 2 of 16 clinically localized prostate cancer s and 17 of 33 metastatic prostate cancers exhibited loss of the miR-101-1 locus (FIG. 9B). Similarly, 4 of 16 clinically localized prostate cancers and 8 of 33 metastatic prostate cancers displayed loss of miR-101-2 (FIG. 9B). FIG. 9C displays a heat-map representation of matched samples in which miR-101 transcript, EZH2 transcript, miR-101-1 genomic loci, and miR-101-2 genomic loci were monitored. EZH2 transcript levels were inversely associated with miR-101 transcript levels across prostate cancer progression to metastasis (P<0.0001). EZH2 tended to be uniformly elevated in samples in which the miR-101-1 or miR-101-2 genomic loci exhibited a loss in copy number (P=0.004, permutation test).

To formally demonstrate that genomic loss of miR-101 loci was somatic in nature, the inventors identified nine metastatic prostate cancers that exhibited loss of miR-101-1 and obtained DNA from matched normal tissue. As expected, eight of nine cases exhibited a marked decrease in relative levels of miR-101-1 copy number in the cancer as compared with that in matched normal tissue (FIG. 9D). The inventors also explored miR-101 genomic loss in other tumor types. Using a number of experimental platforms, the inventors demonstrated focal loss (˜20 kB) of miR-101-1 in a subset of breast, gastric, and prostate cancers. Furthermore, the inventors explored public-domain high-density array comparative genomic hybridization and single-nucleotide polymorphism array data sets and observed a genomic loss of one or both miR-101 loci in a subset of glioblastoma multiforme, lung adenocarcinoma, and acute lymphocytic leukemia.

miR-101, by virtue of its regulation of EZH2, may have profound control over the epigenetic pathways that are active not only in cancer cells but in normal pluripotent embryonic stein cells. Overexpression of miR-101 may configure the histone code of cancer cells to that associated with a more benign cellular phenotype. Because the loss of miR-101 and concomitant elevation of EZH2 are most pronounced in metastatic cancer, the inventors postulate that miR-101 loss may represent a progressive molecular lesion in the development of more aggressive disease. Approaches to reintroduce miR-101 into tumors may have therapeutic benefit by reverting the epigenetic program of tumor cells to a more normal state.

Many modifications and variation of the invention as hereinbefore set forth can be made without departing from the spirit and scope thereof and therefore only such limitations should be imposed as are indicated by the appended claims.

All patent and literature references cited in the present specification are hereby incorporated by reference in their entirety.

### Tables

TABLE 1
QPCR Primers sequences used for monitoring
transcript expression.
Gene

| name | Forward primer | Reverse primer |
|---|---|---|
| EZH2 | TGCAGTTGCTTCAGTACCCA | ATCCCCGTGTACTTTCCCATC |
| | TAAT | ATAAT |
| ADRB2 | TTCCTCTTTGCATGGAATT | AGAGGAGTGGGGGAAGAGTC |
| | TG | |
| hDAB2IP | TGGACGATGTGCTCTATGCC | GGATGGTGATGGTTTGGTAG |
| RUNX3 | TCTGTAAGGCCCAAAGTGGG | ACCTCAGCATGACAATATGTC |
| | TA | ACAA |
| CIITA | CCGACACAGACACCATCAAC | CTTTTCTGCCCAACTTCTGC |
| CDH1 | GGAGGAGAGCGGTGGTCAAA | TGTGCAGCTGGCTCAAGTCAA |
| GAPDH | TGCACCACCAACTGCTTAGC | GGCATGGACTGTGGTCATGAG |

TABLE 2
RQ estimation to determine threshold for
single copy loss in male genomic DNA

| Gene location | genomic region | RQ |
|---|---|---|
| chr1: 65,296,708-65,296,562 | miR101-1 | 1.14 |
| chr2: 56,063,616-56,063,502 | miR217 | 1.37 |
| chrX: 45490429-45490738 | miR221 | 0.46 |
| chrX: 45491265-45491574 | miR222 | 0.56 |
| chrX: 133508210-133508507 | miR424 | 0.56 |
| chrX: 113964173-113964483 | miR448 | 0.63 |
| chrX: 133507924-133508194 | miR503 | 0.67 |
| chrX: 137577438-137577720 | miR504 | 0.56 |
| chrX: 118664629-118664939 | miR766 | 0.66 |
| chrX: 77247072-77247271 | PGK1 | 0.54 |
| chrX: 77247822-77248021 | PGK2 | 0.48 |
| chrX: 77248872-77249071 | PGK3 | 0.55 |
| chr6: 170720579-170720690 | TBP | 1 |

TABLE 3
Primer sequences used for genomic PCR assays

| | | |
|---|---|---|
| miR101-1 | GTACTGTGATAACTGAAGGA | ATTCTGCTTCTCTTTGCCTT |
| | TG | GT |
| miR101-2 | GACTGAACTGTCCTTTTTCGG | CCTTTCTCAATGTGATGGCA |
| miR217 | CTAATGCATTGCCTTCAGCA | TTAGCATCTTGGGCTCACCT |
| miR424 | ACCTGGTGGCAGGAACAC | TGAGGCGCTGCTATACCC |
| miR503 | CAGGCGATGGCCTAAGACT | CAGGGTAAGTCTGGGACTGC |
| miR766 | TGAAGACTCTGGGGACTTTTG | AATATACACAGAGGATTGCTT |
| | | AGCC |
| miR448 | TGGCTGGTTGCATATGTAGG | TGGTGTTTCTGGTGTCTGTCA |
| miR384 | AAAACAAATGTTGCAATCCA | TGCAAATAACAAGATGCCTGA |
| | AA | |
| miR222 | ACTGAGCCATTGAGGGTACC | CCCCAGAAGGCAAAGGAT |
| | TA | |
| miR221 | GTGAGACAGCCAATGGAGAAC | TGTTCGTTAGGCAACAGCTA |
| | | CA |
| miR934 | CAGCCTTTGATGGTGTGTGT | TCCATTACTGGAGACTCTGGG |
| TBP | TTAGCTGGCTCTGAGTATGAA | GCTGGAAAACCCAACTTCTG |
| | TAAC | |

TABLE 4
QPCR Primer sequences used for chromatin
immunoprecipitation.

| Gene | | |
|---|---|---|
| name | Forward primer | Reverse primer |
| ADRB2 | GTGACTTTATGCCCCTTTAGA | GAAGGGCTACAACTGGAACTG |
| | GACAA | GAATA |
| DAB2IP | ATTCCTCCAGGTGGGTGTGG | CTAAGCCGCTGTTGCCTTGGC |
| CIITA | TCCTGGCCCGGGGCCTGG | CTGTTCCCCGGGCTCCCGC |
| RUNX3 | TGTCCCGGGATCCTCTTCT | TAGAGACGTTGGTGCGGAAAT |
| CDH1 | TAGAGGGTCACCGCGTCTAT | TCACAGGTGCTTTGCAGTTC |
| WNT1 | GTTTCTGCTACGCTGCTGCT | CACCAGCTCACTTACCACCA |
| GAPDH | TACTAGCGGTTTTACGGGCG | TCGAACAGGAGGAGCAGAGAG |
| | | CGA |
| KIAA0066 | CTAGGAGGGTGGAGGTAGGG | GCCCCAAACAGGAGTAATGA |
| NUP214 | CAGTGAGGTCTCAGCATCAG | CTGGAGGCTATGGGGGTACT |
| | CA | TG |

### REFERENCES

1. Sparmann, A. and van Lohuizen, M. Nat Rev Cancer, 6: 846-856, 2006.
2. Pasini, D., et al. Cell Cycle, 3: 396-400, 2004.
3. Kleer, C. G., et al. Proc Natl Acad Sci USA, 100: 11606-11611, 2003.
4. Bracken, A. P., et al. Embo J, 22: 5323-5335, 2003.
5. Raman, J. D., et al. Clin Cancer Res, 11: 8570-8576, 2005.
6. Varambally, S., et al. Nature, 419: 624-629, 2002.
7. Rhodes, D. R., et al. Proc Natl Acad Sci USA, 101: 9309-9314, 2004.
8. Kondo, Y., et al. Nat Genet, 40: 741-750, 2008.
9. Gal-Yarn, E. N., et al. Proc Natl Acad Sci USA, in press.
10. Yu, J., et al. Cancer Res, 67: 10657-10663, 2007.
11. Jones, P. A. and Baylin, S. B. Cell, 128: 683-692, 2007,
12. Valencia-Sanchez, M. A., et al. Genes Dev, 20: 515-524, 2006.
13. Lim, L. P., et al. Nature, 433: 769-773, 2005.
14. Ambros, V. Nature, 431: 350-355, 2004.
15. Esquela-Kerscher, A. and Slack, F. J. Nat Rev Cancer, 6: 259-269, 2006.
16. Hermeking, H. Cell, 12: 414-418, 2007.
17. Calin, G. A. and Croce, C. M. Nat Rev Cancer, 6: 857-866, 2006.
18. Saito, Y., et al. Cancer Cell, 9: 435-443, 2006.
19. Robertson, K. D. and Jones, P. A. Oncogene, 18: 3810-3820, 1999.
20. Kim, T. Y., et al. Cancer Res, 66:7490-7501, 2006.
21. Datta, J., et al., Cancer Res, 68: 5049-5058, 2008.
22. Fabbri, M., et al. Proc Natl Acad Sci USA, 104: 15805-15810, 2007.
23. Iorio, M. V., et al. Cancer Res, 65: 7065-7070, 2005.
24. Yanaihara, N., et al. Cancer Cell, 9: 189-198, 2006.
25. Ragnarsson, G., et al. Br J Cancer, 79: 1468-1474, 1999,
26. Wolff, E. M., Liang, G., and Jones, P. A. Nat Clin Pract Urol, 2: 502-510, 2005.
27. Lewis, B. P., et al. Cell, 115: 787-798, 2003,
28. Lee, E. R., et al. Stem Cells, 25: 2191-2199, 2007.
29. Bar, M., et al. Stem Cells, 2008.
30. Lakshmipathy, U., et al. Stem Cells Dev, 16: 1003-1016, 2007.
31. Sinkkonen, L., et al. Nat Struct Mol Biol, 15: 259-267, 2008.
32. Benetti, R., et al. Nat Struct Mal Biol, 15: 268-279, 2008.
33. I. M. Bachmann et al., J. Clin. Oncol. 24, 268 (2006).
34. S. Weikert et al., Int. J. Mol. Med. 16, 349 (2005).
35. Y. Matsukawa et al., Cancer Sci. 97, 484 (2006).
36. Su H, et al. Cancer Res 2009; 69; 1135-42.
37. Friedman J M, et al. Cancer Res 2009; 69:2623-9,
38. Bar M, et al. Stem Cells 2008; 26:2496-505.
39. L. A. Boyer et al., Nature 441, 349 (2006).
40. T. I. Lee et al., Cell 125, 301 (2006).
41. F. Sher at al., Stem Cells 26, 2875 (2008).
42. S. Erhardt et al., Development 130, 4235 (2003).
43. K. Plath et al., Science 300, 131 (2003).
44. R. Cao et al., Science 298, 1039 (2002).
45. A. Kuzmichev, K. Nishioka, H. Erdjument-Bromage, P. Tempst, D. Reinberg, Genes Dev. 16, 2893 (2002).
46. J. Yu et al., Cancer Cell 12, 419 (2007).
47. A. Krek et al., Nat. Genet. 37, 495 (2005),
48. B. John et al., PLoS Biol. 2, e363 (2004).
49. V. Rusinov, V. Baev, I. N. Minkov, M. Tabler, Nucleic Acids Res. 33, W696 (2005).
50. W. Fiskus et al., Mal. Cancer Ther. 5, 3096 (2006).
51. B. P. Lewis, C. B. Burge, D, P. Bartel, Cell 120, 15 (2005).
52. J. Krutzfeldt et al., Nature 438, 685 (2005).

1. A method of inhibiting cell proliferation and colony formation of cancer cells comprising increasing the expression of miRNA in the cells, wherein the miRNA is one or more miRNAs selected from the group consisting of miR-1, miR-143, miR-145, miR-29c, miR-127 miR-224, miR182, and miR-183, miR-196a, miR-10a, or miR-203.

2. The method of claim 1, wherein the cancer cell is bladder, breast, prostate, colon, melanoma, or gastric.

3. (canceled)

4. A composition for inhibiting cell proliferation and colony formation of cancer cells comprising miRNA and the cancer cell, wherein the miRNA is one or more miRNAs selected from the group consisting of miR-1, miR-143, miR-145, miR-29c, miR-127 miR-224, miR182, and miR-183, miR-196a, miR-10a, or miR-203.

5. The composition of claim 4, wherein the cancer cell is bladder, breast, lung, prostate, colon, melanoma, or gastric.

6-9. (canceled)

10. A method of inhibiting tumor formation in a subject comprising increasing the expression of miRNA in the tumor cells of the subject, wherein the miRNA is one or more miRNAs selected from the group consisting of miR-1, miR-101, miR-143, miR-145, miR-29c, miR-127 miR-224, miR182, and miR-183, miR-196a, miR-10a, or miR-203.

11. The method of claim 10, wherein the cancer cell is bladder, breast, lung, prostate, colon, melanoma, or gastric.

12. (canceled)

13. A method of decreasing EZH2 over-expression in a cancer cell comprising increasing the expression of miRNA in the cancer cell, wherein the miRNA is one or more miRNAs selected from the group consisting of miR-1, miR-101, miR-143, miR-145, miR-29c, miR-127 miR-224, miR182, and miR-183, miR-196a, miR-10a, or miR-203.

14. The method of claim 13, wherein the cancer cell is bladder, breast, lung, prostate, colon, melanoma, or gastric.

15. (canceled)

16. A method of inhibiting cell proliferation and colony formation of cancer cells comprising increasing the expression of miR-101 in the cells.

17. A method of decreasing EZH2 over-expression in a cancer cell comprising increasing the expression of miR-101 in the cancer cell.
